(19) United States
(12) Patent Application Publication
(10) Pub. No.: US 2022/0215910 A1
(43) Pub. Date: Jul. 7, 2022

(54) SYSTEM AND METHODS FOR MANAGING HEALTHCARE RESOURCES

(71) Applicant: Cornell University, Ithaca, NY (US)

(72) Inventor: Mary E. CHARLSON, New York, NY (US)

(21) Appl. No.: 17/475,712

(22) Filed: Sep. 15, 2021

Related U.S. Application Data
(63) Continuation of application No. 15/512,622, filed on Mar. 20, 2017, now abandoned.

(51) Int. Cl. G16H 10/60; G16H 40/20; G16H 70/60; G06Q 40/08; G16H 50/30; G16H 40/63; G06F 21/62

(52) U.S. Cl. CPC G16H 10/60; G16H 40/20; G16H 70/60; G06F 21/6245; G16H 50/30; G16H 40/63; G06Q 40/08

(57) ABSTRACT

The present invention is directed to a system and a non-transitory computer readable medium for managing use of hospitalization healthcare resources. Specifically, the embodiments of the invention may accept information from one or more sources, select a population unit, define one or more population units according to enhanced comorbidity, assign one or more values to the one or more population units defined according to enhanced comorbidity, and generate a graphical user interface on which one or more values may be depicted. Some embodiments may be directed to a system and methods for determining whether one or more population units may exceed a use of hospitalization healthcare resources threshold.

### CROSS REFERENCE TO RELATED PATENTS

This application is a continuation application of U.S. patent application Ser. No. 15/512,622 filed Mar. 20, 2017, which is a U.S. National Application of PCT/US15/052946 filed Sep. 29, 2015, which claims the benefit of U.S. Provisional Patent Application No. 62/056,820 filed Sep. 29, 2014, which is incorporated by reference.

### FIELD OF THE INVENTION

The invention relates generally to managing the use of healthcare resources. More specifically, the invention is directed to a system and methods by which individuals and groups who may be at risk of incurring high future healthcare costs and who are at risk of hospitalization, repeated hospitalization and readmission may be predicted. Advantageously, through the use of the invention, resources may be better managed and patients who may more likely benefit from specific intervention may be identified.

### BACKGROUND OF THE INVENTION

Healthcare services are those that are rendered generally to diagnose, treat, and/or prevent the disease, illness, injury, and/or other physical and mental impairments of individuals. Multiple entities—including healthcare professionals, hospitals, health insurance providers, non-profit groups, private institutions, and/or government entities—provide, sustain, and/or support the delivery of healthcare services through the use of a variety of healthcare resources. The ability of these multiple entities to effectively deliver healthcare services may be affected by a variety of geographic, demographic, social, political, and/or economic factors. Some of these factors may change overtime. Some changes may lead to a change in the use of healthcare resources.

The use of healthcare resources may be managed by accounting for hospitalizations, repeated hospitalizations, readmissions, outpatient services, laboratory services, pharmacology services, ambulatory services, and healthcare costs. It may, however, be more difficult to manage the use of healthcare resources by accounting for all healthcare resources because some may have a greater effect on the use of healthcare resources than others. Specifically, hospitalizations, repeated hospitalizations, readmissions, and healthcare costs have a greater effect on the use of healthcare resources. For the purposes of this invention, hospitalizations, repeated hospitalizations, readmissions, and healthcare costs may be termed hospitalization healthcare resources.

Predicting the use of hospitalization healthcare resources including healthcare costs, risk of hospitalization, repeated hospitalization, and readmission may provide benefit to the multiple entities involved in the delivery of healthcare services. Certain government entities may subsidize the delivery of healthcare to their governed constituents. Predicting the use of hospitalization healthcare resources may prevent over subsidizing and/or under subsidizing. Predicting the use of hospitalization healthcare resources may benefit private institutions and/or health insurance providers that help support the costs of healthcare for their beneficiaries. In reaction to these predictions, private institutions and/or health insurance providers may, for example, organize wellness accountability programs or adjust beneficiaries' contributions. In a clinical setting, predicting the use of hospitalization healthcare resources may allow healthcare professionals and/or hospitals to develop intervention strategies to manage healthcare costs, hospitalizations, repeated hospitalizations, and readmission.

Many methods have been developed in attempt to predict the use of hospitalization healthcare resources. Some of these methods focus on individuals to determine whether individuals may be at risk for future high healthcare costs, hospitalizations, repeated hospitalizations and readmission. Some of these methods include steps of accepting information about the individuals from sources, processing the information accepted, and providing an output which may predict the use of hospitalization healthcare resources.

Some known methods that include a step for accepting information from sources further include accepting information from prior medical records and/or prior insurance claims. These sources, however, can provide information largely at a time after a patient has been treated at a clinic. This information may be limited because it may be historic, outdated, and/or may not be current. In addition, these sources may store information in a digital and/or computer readable form and may further store information on servers owned by a variety of entities. Servers owned by private entities may require permission for access to the information. The span of time necessary to acquire permission may also cause information to be outdated.

Some methods that include a step for processing information may further include categorizing information based on clinical diagnoses and/or demographics into groups. For example, one group may comprise males between the ages of 18 and 30 with diabetes. Groups may be assigned group codes.

Some methods that include a step for processing information may further include placing group codes into hierarchies based on how likely the hierarchy is to use of hospitalization healthcare resources. In this way, an individual having certain clinical diagnoses and/or demographics may be associated with hierarchies correlating to the use of hospitalization healthcare resources. However, there may be uncertainty in predicting an individual's use of hospitalization healthcare resources if that individual is associated with more than one hierarchy.

Some methods that include a step for processing may further include using a comorbidity index. Generally, a comorbidity index is a list of chronic diseases in which each included chronic disease is assigned a value that predicts a future effect. Values may be weighted according to the likelihood of a future effect occurring. This Index may be used in methods which focus on individuals with multiple chronic diseases. Individuals may be assigned values from the Index based on whether the individuals have chronic diseases listed in the Index. Values may then be combined to predict a likelihood of a future effect for individuals with multiple chronic diseases. For example, the Index may provide scores of 1 for a prior myocardial infarction and 6 for metastatic cancer, but an individual afflicted with both chronic diseases may be assigned a value of 7.

Indices similar to the comorbidity index were developed for specific purposes other than predicting the use of hospitalization healthcare resources. For example, indices included values weighted according to a risk that an individual will die. Also, indices may not have predicted future costs, future hospitalizations, repeated hospitalizations and readmissions. Methods which use these indices may introduce uncertainty in predicting whether individuals may use hospitalization healthcare resources by not targeting the individuals with chronic diseases most associated with high use of hospitalization healthcare resources.

Some known methods that include a step for providing an output for predicting the use of hospitalization healthcare resources may further include providing group codes, hierarchy codes, and/or currency values. Group codes, and/or hierarchy codes may not be readily usable by healthcare entities because they are not clinically interpretable or mappable by a person unfamiliar with the codes. Even for people familiar with the codes, it may be difficult to immediately associate codes with the use of hospitalization healthcare resources. A graphical user interface displaying currency values may be too specific. Specific currency values may be misleading and introduce uncertainty in estimating the use of hospitalization healthcare resources.

There is a need for a new test for predicting the use of hospitalization healthcare resources for both adults and children which may accept information from wider variety of sources, process the information in more simplified steps, and provide a more comprehensible output. The present invention satisfies these needs.

### SUMMARY OF THE INVENTION

The present invention may be directed to a system and methods that may use non-transitory computer readable medium for predicting the use of hospitalization healthcare resources. Specifically, certain embodiments of the present invention may accept information from one or more sources, select one or more population units from the information to provide a selected group, define one or more population units from the selected group according to enhanced comorbidity to provide an enhanced comorbidity group, assign one or more values to one or more population units from the enhanced comorbidity group, and generate a graphical user interface on which one or more values may be depicted.

In certain preferred embodiments, the present invention includes using an enhanced comorbidity index. The enhanced comorbidity index may be a comorbidity index which lists specific chronic diseases weighted according to a likelihood for the use of hospitalization healthcare resources. The enhanced comorbidity index may account for a wide range of physical and mental conditions including inflammatory bowel disease, major mental illnesses, and/or organ transplants. Enhanced comorbidity may also include conditions which affect children such as autism. The predicted effect of these specific chronic diseases on the use of hospitalization healthcare resources may also be termed for the purposes of this invention as “enhanced comorbidity”. Enhanced comorbidity may be assigned to patients regardless of age—that is, to both adults and children. Also, enhanced comorbidity may be used to predict future costs, future hospitalizations, repeated hospitalizations, and readmissions.

Embodiments of the present invention may instruct a processor to accept information from a wider variety of sources. Sources of information that may be accepted and processed by the system may include an interview, a questionnaire, medical records, a registry or repository, and/or insurance claim data. Sources may provide information in analog form or in digital form. The sources may provide information including personal information, anonymous information, diagnostic information, demographic information, and/or geographic information.

Advantageously, embodiments of the present invention may process and provide the information in more simplified steps. In some embodiments, a processor may be instructed to select one or more population units from the information to provide a selected group; define one or more population units from the selected group according to enhanced comorbidity to provide an enhanced comorbidity group; and assign one or more values to one or more population units from the enhanced comorbidity group.

One or more population units may be selected based on information including personal information, anonymous information, diagnostic information, demographic information and/or geographic information of one or more individuals. One or more population units may be defined according to enhanced comorbidity based on whether information indicates one or more individuals comprising the one or more population units have one or more chronic diseases listed in an enhanced comorbidity index. Values may be assigned to one or more population units defined according to enhanced comorbidity based on whether the one or more chronic diseases are listed in an enhanced comorbidity index.

Embodiments of the invention may provide a more comprehensible output. In some embodiments, a processor may be instructed to generate a graphical user interface on which one or more values may be depicted. The one or more values may be numbers weighted according to the use of hospitalization healthcare resources. It may be readily apparent from viewing the one or more values depicted whether one or more population units may use of hospitalization healthcare resources.

Alternative embodiments of the present invention may be useful in determining whether one or more population units exceeds a use of hospitalization healthcare resources threshold. In some embodiments, a processor may be instructed to request a use of hospitalization healthcare resources threshold; compare one or more values to the use of hospitalization healthcare resources threshold; and generate a graphical user interface on which a determination of whether one or more population units exceed the use of hospitalization healthcare resources threshold may be depicted. A use of hospitalization healthcare resources threshold may be identified as value associated with use of hospitalization healthcare resources, and may be identified based on the range of weighted values listed in an enhanced comorbidity index.

One object of the present invention may be to provide a new test for managing the use of hospitalization healthcare resources including healthcare costs, hospitalizations, repeated hospitalizations, and readmissions.

A further object of the invention may be to predict whether one or more individuals may use of hospitalization healthcare resources.

Another object of the present invention may be to determine whether one or more population units exceed a use of hospitalization healthcare resources threshold.

An additional object of this invention may be to provide a new test capable of more accurately predicting whether one or more individuals may use of hospitalization healthcare resources in the usual clinical context.

It may be an object of this invention to provide a new test capable of more accurately predicting whether one or more individuals may use of hospitalization healthcare resources for new health insurance beneficiary enrollees.

The present invention, its attributes and advantages may be further understood with reference to the detailed description of presently contemplated embodiments, taken in conjunction with the accompanying drawings.

### BRIEF DESCRIPTION OF THE DRAWINGS

The preferred embodiments of the invention may be described in conjunction with the appended drawings provided to illustrate and not to limit the invention, where like designations denote like elements, and in which:

FIG. 1 illustrates a flowchart of steps that an embodiment of the present invention causes a processor to perform.

FIG. 2 illustrates an enhanced comorbidity index that displays values weighted according to the use of hospitalization healthcare resources for corresponding chronic diseases.

FIG. 3 illustrates a flowchart of steps that an alternative embodiment of the present invention causes a processor to perform.

FIG. 4 illustrates an exemplary graphical user interface on which the value for an individual is depicted.

FIG. 5 illustrates an exemplary graphical user interface on which a determination of whether an individual exceeds a use of hospitalization healthcare resources threshold is depicted.

FIG. 6 illustrates an exemplary graphical user interface on which an population unit's use of hospitalization healthcare resources may be depicted for an institution.

FIG. 7 illustrates an exemplary graphical user interface on which a population unit's repeated hospitalization may be depicted for an institution.

FIG. 8 illustrates an exemplary graphical user interface on which a population unit's readmissions may be depicted for an institution.

FIG. 9 illustrates a diagram of an embodiment of a cloud computing system.

FIG. 10 illustrates a diagram of an embodiment of a computer system.

### DETAILED DESCRIPTION

In the following description, for purposes of explanation, specific numbers, materials, and configurations are set forth in order to provide a thorough understanding of the invention. It may be apparent, however, to one having ordinary skill in the art that the invention may be practiced without these specific details. In some instances, well-known features may be omitted or simplified so as not to obscure the present invention. Furthermore, reference in the specification to “one embodiment” or “an embodiment” means that a particular feature, structure or characteristic described in connection with the embodiment is included in at least one embodiment of the invention. The appearances of the phrase “in one embodiment” in various places in the specification are not necessarily all referring to the same embodiment.

The present invention may be directed to a system and methods for managing the use of hospitalization healthcare resources. In certain preferred embodiments, the present invention relates to a non-transitory computer-readable storage medium storing an executable program. The executable program may cause one or more processors to execute a method comprising the steps: accept information from one or more sources; select one or more population units from the information to provide a selected group; define one or more population units from the selected group according to enhanced comorbidity to provide an enhanced comorbidity group; assign one or more values to one or more population units from the enhanced comorbidity group; and generate a graphical user interface on which one or more values may be depicted to predict the use of hospitalization healthcare resources.

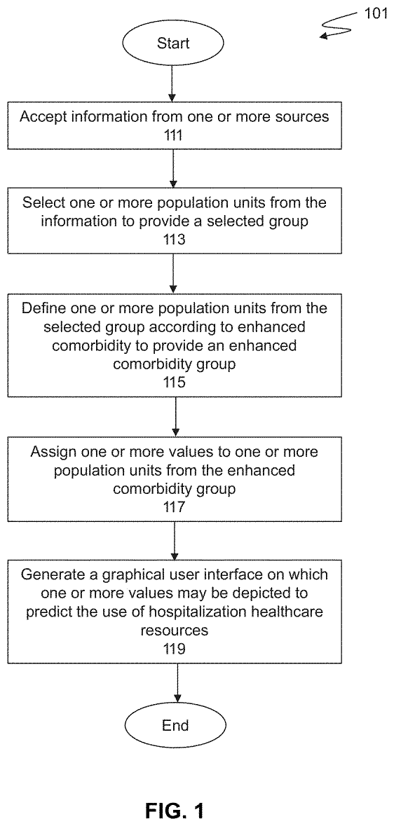

In certain preferred embodiments, the system may comprise at least non-transitory computer-readable storage media, one or more processors, and one or more displays. FIG. 1 illustrates a flowchart of an embodiment of the system 101. This embodiment may accept information from one or more sources 111, may select one or more population units from the information to provide a selected group 113, may define one or more population units from the selected group according to enhanced comorbidity to provide an enhanced comorbidity group 115, may assign one or more values to one or more population units from the enhanced comorbidity group 117, and may generate a graphical user interface on which one or more values may be depicted to predict the use of hospitalization healthcare resources 119.

In some embodiments, the non-transitory computer-readable storage medium storing an executable program may cause one or more processors to accept information from one or more sources 111. The one or more processors may accept information by receiving information in digital form and storing the information on non-transitory computer-readable storage media. In addition, the one or more processors may accept information by transforming information from an analog form to a digital form and storing the transformed information on non-transitory computer-readable storage media. Further, the one or more processors may transform analog information into digital information by receiving information from input devices including a scanner, keyboard, touch screen, joystick, trackball, mouse, monitor, microphone, camera or any combination thereof, capable of input.

Some embodiments of the present invention may accept information from one or more sources 111 including an interview with an individual, a questionnaire, medical records, a registry or repository, and/or insurance claim data.

Information may be accepted from an interview such as by verbally requesting one or more individuals to identify chronic diseases and/or symptoms of chronic diseases in analog form, and causing one or more processors to transform the information into digital form. In addition, information may be accepted from an individual in digital form by causing a processor to receive the information and store the information on non-transitory computer-readable storage media.

Information may be accepted from a questionnaire by requesting one or more individuals to identify chronic diseases, and/or symptoms of chronic diseases in analog form, and causing a processor or to transform the information into digital form. In addition, the questionnaire may be displayed to one or more individuals in digital form, and request one or more individuals to identify chronic diseases, and/or symptoms of chronic diseases in digital form.

Information may be accepted from medical records by causing a processor to transform information in analog form, contained in medical records, into information in digital form. In addition, information may be accepted from medical records in digital form by causing a processor to receive the information and store the information on non-transitory computer-readable storage media.

Information may be accepted from registry or repository data by causing a processor to transform information in analog form, contained in a registry or repository, into information in digital form. In addition, information may be accepted from a registry or repository in digital form by causing a processor to receive the information and store the information on non-transitory computer-readable storage media.

Information may be accepted from insurance claim data by causing a processor to transform information in analog form, contained in insurance claim data, into information in digital form. In addition, information may be accepted from medical records in digital form by causing a processor to receive the information and store the information on non-transitory computer-readable storage media.

Sources may provide information about one or more individuals including personal information, anonymous information, diagnostic information, demographic information, and/or geographic information. Personal information may be information from which the identity of a person may be readily apparent including a name, a residency address, a public identification number, and/or a private identification number. For example, the identity of a person may be readily apparent from that person's name.

Anonymous information may be information from which the identity of a person may not be readily apparent. For example, the identity of a person may not be readily apparent from a number assigned to maintain that person's anonymity. Sources may associate personal information with anonymous information. For example, a source may assign the anonymous information number “20120” to personal information name “John Smith”. Sources may restrict access to personal information, and/or allow access to anonymous information. Sources may associate information of chronic diseases with personal information and/or anonymous information.

In some embodiments, diagnostic information may include information identifying chronic diseases. For example, information provided by a medical chart may identify a patient has “diabetes”. Diagnostic information may also include information symptomatic of chronic diseases. For example, information provided by a medical chart may identify a patient has “motor delay”.

In some embodiments, demographic information may include information identifying age, sex, ethnicity, place of employment, healthcare providers, health insurance providers, and health insurance plans. Geographic information may include one or more individuals' residences, site of care, and/or site of work.

FIG. 1 further illustrates a system 101 comprising non-transitory computer-readable storage medium storing an executable program that may cause one or more processors to select one or more population units from the information to provide a selected group 113. A population unit may comprise one or more individuals. To select a population unit the processor may select a one or more individuals based on personal information, anonymous information, diagnostic information, demographic information, and/or geographic information.

In some embodiments the processor may select a population unit based on personal information and/or anonymous information. For example, the processor may select a population unit comprising the individual based on the information “John Smith”. In another example, the processor may select a population unit comprising the individuals “John Smith”, “Paul Jones”, and “Jane Doe”. In a further example, the processor may select a population unit comprising the anonymous individuals “20120”, “21501”, and “36789”.

In some embodiments, the processor may select a population unit based on diagnostic information. For example, the processor may select a population unit comprising the individual having “autism, diabetes, and motor delay”.

In some embodiments, the processor may select a population unit based on demographic information. For example, the processor may select individuals which are male. In another example, the processor may select individuals which are male and below the age of 18. In yet another example, the processor may select individuals employed by “Metro Hospital”.

In some embodiments, the processor or may select a population unit based on geographic information. For example, the processor may select individuals residing in “Blackacre county”. In another embodiment, the processor may select individuals having residing in “State of Marshall”.

FIG. 1 further illustrates a system 101 comprising non-transitory computer-readable storage medium storing an executable program that may cause one or more processors to define one or more population units from the selected group according to enhanced comorbidity to provide an enhanced comorbidity group 115. The one or more processors may define the one or more population units according to enhanced comorbidity based on the chronic diseases each individual has. The one or more processors may define according to enhanced comorbidity based on information accepted from the same source. For example, information identifying an individual accepted from a medical record may be defined according to enhanced comorbidity based on information identifying chronic diseases from that same medical record. In addition, the one or more processors may also define according to enhanced comorbidity based on information from different sources.

For example, information identifying an individual accepted from a medical record may be defined according to enhanced comorbidity based on information identifying an individual and information identifying chronic diseases accepted from a questionnaire. The processor may define according to enhanced comorbidity based on information accepted for one or more individuals for a population unit. For example, the processor may define according to enhanced comorbidity based on information identifying an individual and information identifying chronic diseases of that individual with information identifying a different individual and information identifying chronic diseases of that different individual.

FIG. 1 further illustrates a system 101 comprising non-transitory computer-readable storage medium storing an executable program that may cause one or more processors to assign one or more values to one or more population units from the enhanced comorbidity group 117. The one or more processors may assign values to each of one or more individuals comprising one or more population units defined according to enhanced comorbidity based on chronic diseases listed in an enhanced comorbidity index. The processor assigns values corresponding to each chronic disease listed in the enhanced comorbidity index.

For example, if an enhanced comorbidity index lists diabetes as a chronic disease and diabetes corresponds to a value of 1, then each individual that has diabetes in a population unit may be assigned a value of 1. In addition, if one or more individuals have more than one chronic disease, then the one or more individuals may be assigned values corresponding to each chronic disease listed in an enhanced comorbidity index. These values may be combined. For example, if an individual has autism which corresponds to a value of 3, and has diabetes which corresponds to a value of 1, then the individual may be assigned a combined value of 4.

FIG. 1 further illustrates a system 101 comprising non-transitory computer-readable storage medium storing an executable program that may cause one or more processors to generate a graphical user interface on which one or more values may be depicted to predict the use of hospitalization healthcare resources 119. The values depicted may be weighted according to the use of hospitalization healthcare resources. The processor may further select one or more population units from the information to provide a selected group to be depicted by the graphical user interface. The population unit depicted may be an individual or a group of individuals. The graphical user interface may further depict specific chronic diseases corresponding to the values for one or more population units. The method may still further include generating a graphical user interface depicting healthcare costs correlating to the values for one or more population units.

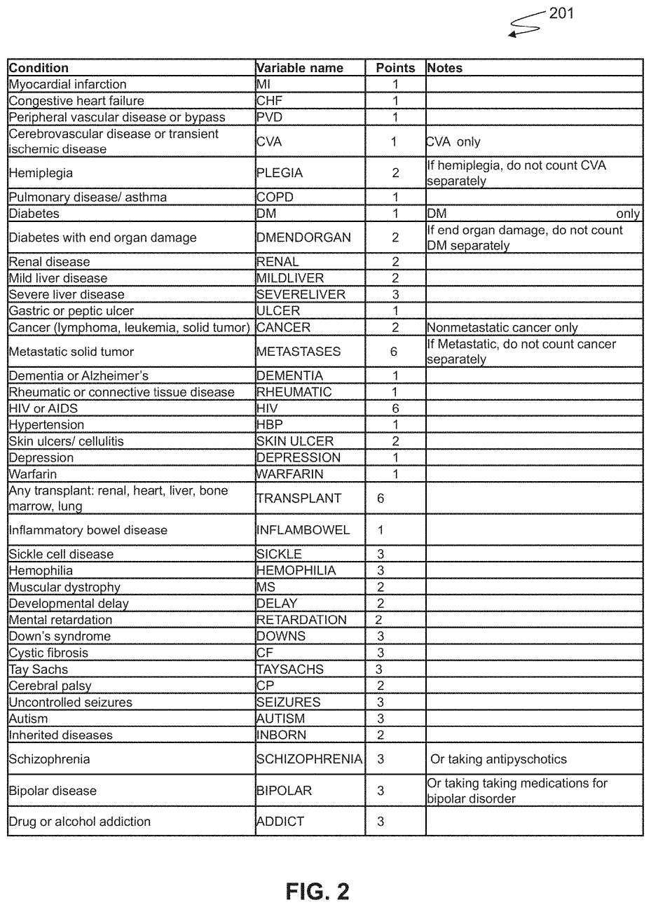

FIG. 2 illustrates an exemplary enhanced comorbidity index 201 that displays values for corresponding chronic diseases. The values corresponding to each chronic disease may be weighted according to the use of hospitalization healthcare resources. The values listed in the enhanced comorbidity index may be 1, 2, 3, and/or 6. A higher value may reflect an increased use of hospitalization healthcare resources compared to a lower value. Values may reflect an increased risk for the use of hospitalization healthcare resources based on the approximate use of hospitalization healthcare resources of necessary hospital, medical, maternity, behavioral health and/or pharmacy services received by individuals having each chronic disease. For example, an individual having myocardial infraction and assigned a value of 1 and a different individual having diabetes and assigned a value of one will have approximately the same healthcare costs of $2,000. The enhanced comorbidity index may be used in assigning values to adults and/or children.

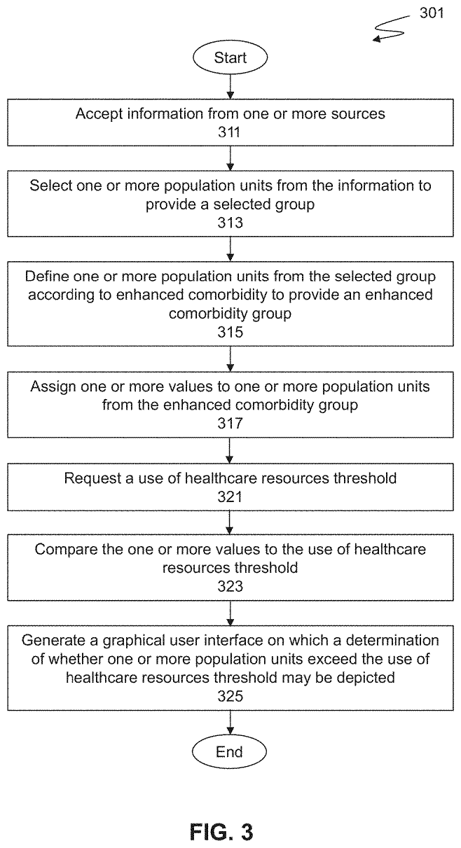

In alternative embodiments, the system may comprise at least non-transitory computer-readable storage media, one or more processors, and one or more displays. FIG. 3 illustrates a flowchart of an embodiment of the system 301. This embodiment may accept information from one or more sources 311, may select one or more population units from the information to provide a selected group 313, may define one or more population units from the selected group according to enhanced comorbidity to provide an enhanced comorbidity group 315, may assign one or more values to one or more population units from the enhanced comorbidity group 317, may request a use of hospitalization healthcare resources threshold 321, may compare the one or more values to the use of hospitalization healthcare resources threshold 323, and may generate a graphical user interface on which a determination of whether one or more population units exceed the use of hospitalization healthcare resources threshold may be depicted 325.

FIG. 3 further illustrates a system 301 comprising non-transitory computer-readable storage medium storing an executable program that may cause one or more processors to request a use of hospitalization healthcare resources threshold 321. The use of hospitalization healthcare resources threshold may be determined in a variety of ways. The use of hospitalization healthcare resources threshold may be determined from prior interviews, questionnaires, medical records, registries or repositories, and/or insurance claim data. Additionally, the use of hospitalization healthcare resources threshold may be identified as value associated with use of hospitalization healthcare resources, and may be identified based on the range of weighted values listed in an enhanced comorbidity index.

For example, a hospital may identify a use of hospitalization healthcare resources threshold of 7 based on an enhanced comorbidity index. In another example, the hospital may identify the use of hospitalization healthcare resources threshold based on an enhanced comorbidity value associated with an approximate estimate of repeated hospitalization “greater than 3 hospitalizations annually”. In another example, a health insurance provider may identify a use of hospitalization healthcare resources of 6 based on an enhanced comorbidity index. In another example, the health insurance provider may identify the use of hospitalization healthcare resources threshold based on an approximate amount of healthcare costs “greater than $4,000 annually”.

FIG. 3 further illustrates a system 301 comprising non-transitory computer-readable storage medium storing an executable program that may cause one or more processors to compare the one or more values to the use of hospitalization healthcare resources threshold 323. For example, the one or more values “5” assigned to an individual “John Smith” may be compared the use of hospitalization healthcare resources threshold “7” identified by “Metro Hospital”.

FIG. 3 further illustrates a system 301 comprising non-transitory computer-readable storage medium storing an executable program that may cause one or more processors to generate a graphical user interface on which a determination of whether one or more population units exceed the use of hospitalization healthcare resources threshold may be depicted 325. For example, the graphical user interface can depict the one or more individuals' values and the use of hospitalization healthcare resources threshold. In another example, the graphical user interface may depict a “pass” or “fail”.

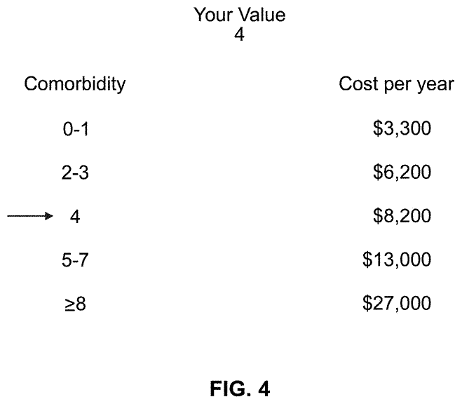

FIG. 4 illustrates an exemplary graphical user interface on which the value for an individual is depicted. In some embodiments, a graphical user interface similar to the one illustrated in FIG. 4 may be used to predict a use of hospitalization healthcare resources for an individual. For example, a user may view a graphical user interface and the correlation between their value and a risk for high healthcare costs. In one embodiment, the graphical user interface may include one column listing enhanced comorbidity values, and one column listing healthcare costs correlating to the values. As illustrated in FIG. 4, a user viewing the graphical user interface may see a value assigned to an individual, and also see the correlation to healthcare costs. In another embodiment, the graphical user interface may include a column listing hospitalizations. In other embodiments, the graphical user interface may include a column listing repeated hospitalizations. In yet another embodiment, the graphical user interface may include a column listing readmissions.

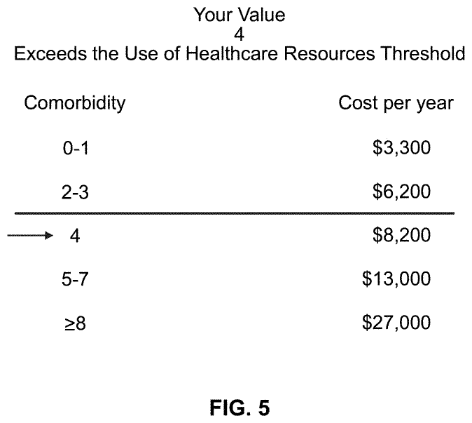

FIG. 5 illustrates an exemplary graphical user interface on which a determination of whether an individual exceeds a use of hospitalization healthcare resources threshold is depicted. In some embodiments, a graphical user interface similar to the one illustrated in FIG. 5 may be used to determine whether an individual exceeds a use of healthcare resources threshold. For example, a user may view a graphical user interface and the correlation between their value and a use of hospitalization healthcare resources threshold.

In one embodiment, the graphical user interface may include one column listing enhanced comorbidity values, and one column listing healthcare costs correlating to the values. As illustrated in FIG. 5, a user viewing the graphical user interface may see a value assigned to an individual, see the correlation to healthcare costs, and see that the individual exceeds a use of healthcare resources threshold. In another embodiment, the graphical user interface may include a column listing hospitalizations. In other embodiments, the graphical user interface may include a column listing repeated hospitalizations. In yet another embodiment, the graphical user interface may include a column listing readmissions.

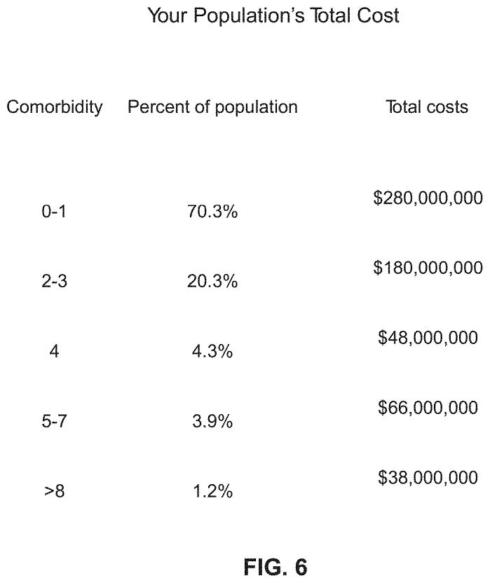

FIG. 6 illustrates an exemplary graphical user interface on which a population unit's use of hospitalization healthcare resources may be depicted for an institution. In some embodiments, a graphical user interface similar to the one illustrated in FIG. 6 may be used to determine a use of hospitalization healthcare resources threshold. For example, a user may view a graphical user interface similar to the one illustrated in FIG. 6 and determine that individuals with a value greater than 4 are at risk for high healthcare costs. In some embodiments, the graphical user interface may include a column listing enhanced comorbidity values, another column with healthcare costs correlating to the values, and another column with the percent of the population. In another embodiment, the graphical user interface may include a column listing hospitalizations. In other embodiments, the graphical user interface may include a column listing repeated hospitalizations. In yet another embodiment, the graphical user interface may include a column listing readmissions.

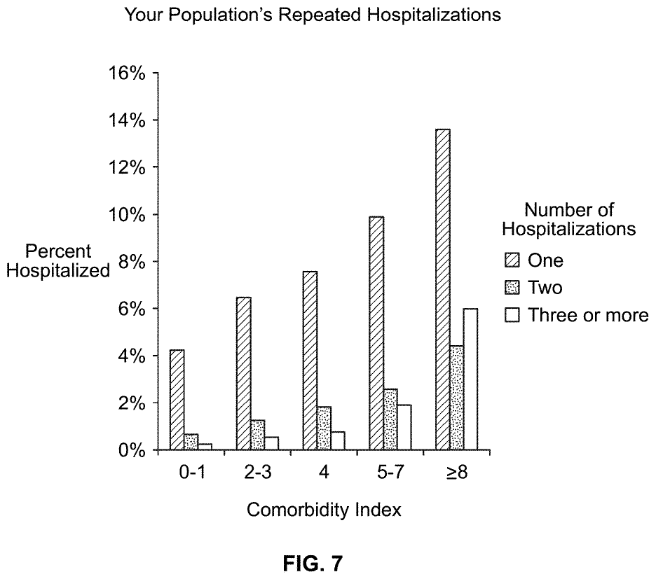

FIG. 7 illustrates an exemplary graphical user interface on which a population unit's repeated hospitalization may be depicted for an institution. In some embodiments, a graphical user interface similar to the one illustrated in FIG. 7 may be used to determine a use of hospitalization healthcare resources threshold. For example, a user may view a graphical user interface similar to the one illustrated in FIG. 7 and determine that individuals with a value greater than 4 are at risk for repeated hospitalizations. Accordingly, when requested, a use of hospitalization healthcare resources threshold of 4 may be provided.

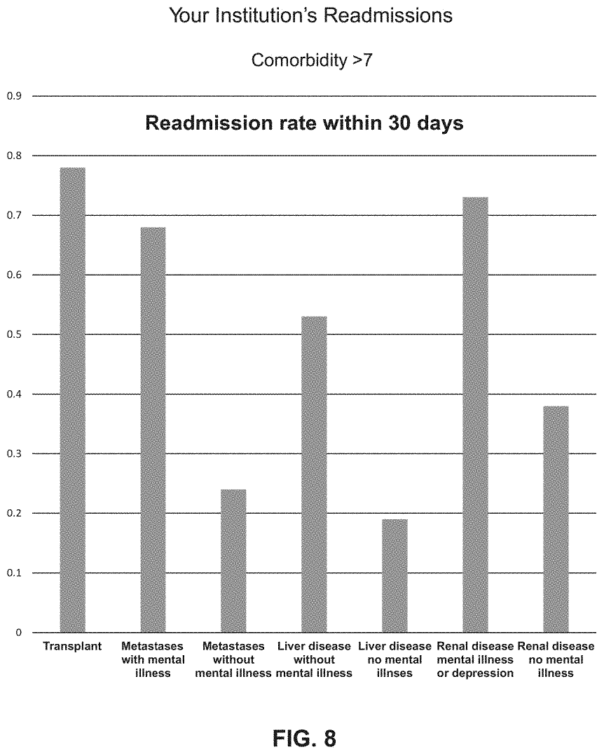

FIG. 8 illustrates an exemplary graphical user interface on which a population unit's readmissions may be depicted for an institution. In some embodiments, a graphical user interface similar to the one illustrated in FIG. 8 may be used to determine whether individuals with a specific chronic disease in a population unit may use hospitalization healthcare resources. For example, a user may view a graphical user interface similar to the one illustrated in FIG. 8 and determine that individuals with metastases or transplants are at risk for readmissions.

In some embodiments, a graphical user interface, similar to the one illustrated in FIG. 8, may display the percentage of one or more population units having comorbidity values over 7, which are at risk for readmission, and the specific chronic diseases the one or more populations units have. In other embodiments, the graphical user interface may display healthcare costs, hospitalization, and/or repeated hospitalization associated with one or more population units with the specific chronic diseases. Accordingly, an intervention plan may be developed to treat patients with the specific chronic disease.

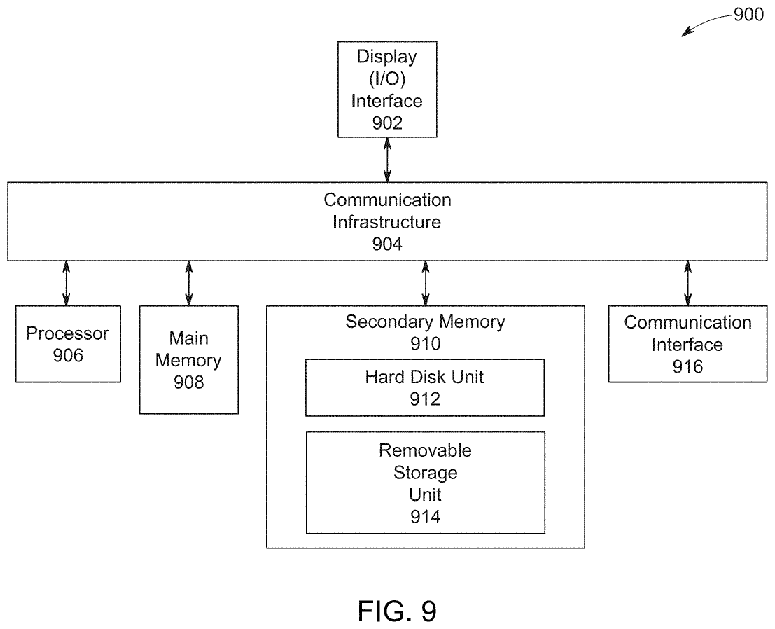

FIG. 9 illustrates a diagram of a system which may be an embodiment of the present invention. Computer system 900 includes an input/output interface 902 connected to communication infrastructure 904—such as a bus—, which forwards data such as graphics, text, and information, from the communication infrastructure 904 or from a frame buffer (not shown) to other components of the computer system 900. The input/output interface 902 may be, for example, a display device, a keyboard, touch screen, joystick, trackball, mouse, monitor, speaker, printer, Google Glass® unit, web camera, any other computer peripheral device, or any combination thereof, capable of entering and/or viewing data.

Computer system 900 includes one or more processors 906, which may be a special purpose or a general-purpose digital signal processor configured to process certain information. Computer system 900 also includes a main memory 908, for example random access memory (RAM), read-only memory (ROM), mass storage device, or any combination thereof. Computer system 900 may also include a secondary memory 910 such as a hard disk unit 912, a removable storage unit 914, or any combination thereof. Computer system 900 may also include a communication interface 916, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 908, secondary memory 910, communication interface 916, or a combination thereof, function as a computer usable storage medium, otherwise referred to as a computer readable storage medium, to store and/or access computer software including computer instructions. For example, computer programs or other instructions may be loaded into the computer system 900 such as through a removable storage device, for example, a floppy disk, ZIP disks, magnetic tape, portable flash drive, optical disk such as a CD or DVD or Blu-ray, Micro-Electro-Mechanical Systems (MEMS), nanotechnological apparatus. Specifically, computer software including computer instructions may be transferred from the removable storage unit 914 or hard disc unit 912 to the secondary memory 910 or through the communication infrastructure 904 to the main memory 908 of the computer system 900.

Communication interface 916 allows software, instructions and data to be transferred between the computer system 900 and external devices or external networks. Software, instructions, and/or data transferred by the communication interface 916 are typically in the form of signals that may be electronic, electromagnetic, optical or other signals capable of being sent and received by the communication interface 916. Signals may be sent and received using wire or cable, fiber optics, a phone line, a cellular phone link, a Radio Frequency (RF) link, wireless link, or other communication channels.

Computer programs, when executed, enable the computer system 900, particularly the processor 906, to implement the methods of the invention according to computer software including instructions.

The computer system 900 described may perform any one of, or any combination of, the steps of any of the methods according to the invention. It is also contemplated that the methods according to the invention may be performed automatically.

The computer system 900 of FIG. 9 is provided only for purposes of illustration, such that the invention is not limited to this specific embodiment. It is appreciated that a person skilled in the relevant art knows how to program and implement the invention using any computer system.

The computer system 900 may be a handheld device and include any small-sized computer device including, for example, a personal digital assistant (PDA), smart hand-held computing device, cellular telephone, or a laptop or netbook computer, handheld console or MP3 player, tablet, or similar handheld computer device, such as an iPad®, iPad Touch® or iPhone®.

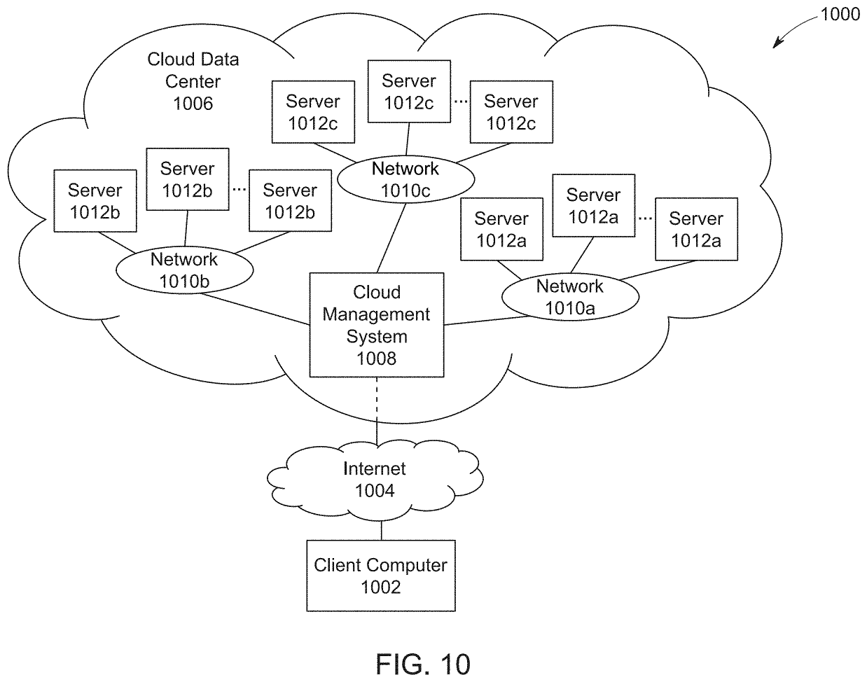

FIG. 10 illustrates an exemplary cloud computing system 1000 that may be an embodiment of the present invention. The cloud computing system 1000 includes a plurality of interconnected computing environments. The cloud computing system 1000 utilizes the resources from various networks as a collective virtual computer, where the services and applications can run independently from a particular computer or server configuration making hardware less important.

Specifically, the cloud computing system 1000 includes at least one client computer 1002. The client computer 1002 may be any device through the use of which a distributed computing environment may be accessed to perform the methods disclosed herein, for example, a traditional computer, portable computer, mobile phone, personal digital assistant, tablet to name a few. The client computer 1002 includes memory such as random access memory (RAM), read-only memory (ROM), mass storage device, or any combination thereof. The memory functions as a computer usable storage medium, otherwise referred to as a computer readable storage medium, to store and/or access computer software and/or instructions.

The client computer 1002 also includes a communications interface, for example, a modem, a network interface (such as an Ethernet card), a communications port, a PCMCIA slot and card, wired or wireless systems, etc. The communications interface allows communication through transferred signals between the client computer 1002 and external devices including networks such as the Internet 1004 and cloud data center 1006. Communication may be implemented using wireless or wired capability such as cable, fiber optics, a phone line, a cellular phone link, radio waves or other communication channels.

The client computer 1002 establishes communication with the Internet 1004—specifically to one or more servers—to, in turn, establish communication with one or more cloud data centers 1006. A cloud data center 1006 includes one or more networks 1010a, 1010b, 1010c managed through a cloud management system 1008. Each network 1010a, 1010b, 1010c includes resource servers 1012a, 1012b, 1012c, respectively. Servers 1012a, 1012b, 1012c permit access to a collection of computing resources and components that can be invoked to instantiate a virtual machine, process, or other resource for a limited or defined duration. For example, one group of resource servers can host and serve an operating system or components thereof to deliver and instantiate a virtual machine. Another group of resource servers can accept requests to host computing cycles or processor time, to supply a defined level of processing power for a virtual machine. A further group of resource servers can host and serve applications to load on an instantiation of a virtual machine, such as an email client, a browser application, a messaging application, or other applications or software.

The cloud management system 1008 can comprise a dedicated or centralized server and/or other software, hardware, and network tools to communicate with one or more networks 1010a, 1010b, 1010c, such as the Internet or other public or private network, with all sets of resource servers 1012a, 1012b, 1012c. The cloud management system 1008 may be configured to query and identify the computing resources and components managed by the set of resource servers 1012a, 1012b, 1012c needed and available for use in the cloud data center 1006. Specifically, the cloud management system 1008 may be configured to identify the hardware resources and components such as type and amount of processing power, type and amount of memory, type and amount of storage, type and amount of network bandwidth and the like, of the set of resource servers 1012a, 1012b, 1012c needed and available for use in the cloud data center 1006. Likewise, the cloud management system 1008 can be configured to identify the software resources and components, such as type of Operating System (OS), application programs, and the like, of the set of resource servers 1012a, 1012b, 1012c needed and available for use in the cloud data center 1006.

The present invention is also directed to computer products, otherwise referred to as computer program products, to provide software to the cloud computing system 1000. Computer products store software on any computer useable medium, known now or in the future. Such software, when executed, may implement the methods according to certain embodiments of the invention. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, optical storage devices, Micro-Electro-Mechanical Systems (MEMS), nanotechnological storage device, etc.), and communication mediums (e.g., wired and wireless communications networks, local area networks, wide area networks, intranets, etc.). It is to be appreciated that the embodiments described herein may be implemented using software, hardware, firmware, or combinations thereof.

The cloud computing system 1000 of FIG. 10 is provided only for purposes of illustration and does not limit the invention to this specific embodiment. It is appreciated that a person skilled in the relevant art knows how to program and implement the invention using any computer system or network architecture.

While the disclosure is susceptible to various modifications and alternative forms, specific exemplary embodiments of the invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

1.-20. (canceled)

21. A predictive health management system comprising:
one or more non-transitory computer-readable storage media bearing computer-readable executable instructions;
one or more output devices; and
one or more processors operatively associated with the one or more output devices and the one or more non-transitory computer-readable storage media, the one or more processors being configured, upon execution of the computer-readable executable instructions, to:
select a group, via the one or more processors, from population information stored on the one or more non-transitory computer-readable storage media, the population information comprising predetermined chronic conditions associated with at least some members of the population;
identify, via the one or more processors, at least one member within the selected group having a sum of predetermined chronic conditions equal to or greater than a selected threshold number of predetermined chronic conditions selected from a set of predetermined chronic conditions comprising a plurality of predetermined chronic conditions, each of the plurality of predetermined chronic conditions having a predefined comorbidity value associated therewith;
determine, via the one or more processors, an enhanced comorbidity value for the at least one member within the selected group, the enhanced comorbidity value relating to a number of predetermined chronic conditions in excess of the threshold number of predetermined chronic conditions and further relating to predefined comorbidity values associated with each of the number of predetermined chronic conditions;
determine, via the one or more processors, a level of use of at least one healthcare resource selected from a set of predefined healthcare resources corresponding to the determined enhanced comorbidity value for the at least one member; and
output to at least one of the one or more output devices, via the one or more processors, a representation of level of use of the at least one healthcare resource corresponding to healthcare services delivery for the at least one member within the selected group.

22. The predictive health management system according to claim 21,
wherein the at least one member within the selected group comprises a plurality of members, and
wherein the one or more processors are configured, upon execution of the computer-readable executable instructions, to:
identify, via the one or more processors, a plurality of members within the selected group having the sum of predetermined chronic conditions equal to or greater than the selected threshold number of predetermined chronic conditions selected from the set of predetermined chronic conditions comprising the plurality of predetermined chronic conditions;
determine, via the one or more processors, an enhanced comorbidity value for the plurality of members within the selected group, the enhanced comorbidity value for the plurality of members within the selected group relating to a number of predetermined chronic conditions in excess of the threshold number of predetermined chronic conditions and further relating to predefined comorbidity values associated with each of the number of predetermined chronic conditions;
determine, via the one or more processors, a level of use of at least one healthcare resource selected from the set of predefined healthcare resources for the plurality of members within the selected group having a determined enhanced comorbidity value based on the determined enhanced comorbidity value for each of the plurality of members within the selected group; and
output to at least one of the one or more output devices, via the one or more processors, a representation of the level of use of the at least one healthcare resource appropriate to healthcare services delivery for the plurality of members within the selected group.

23. The predictive health management system according to claim 21, wherein the population information stored on the one or more non-transitory computer-readable storage media comprises age associated with each member of the population, and wherein the population information includes predetermined chronic conditions associated with at least some members of the population who are adults and includes predetermined chronic conditions associated with at least some members of the population who are children.

24. The predictive health management system according to claim 23, wherein the at least one member within the selected group is associated with population information for a child.

25. The predictive health management system according to claim 23, wherein the at least one member within the selected group is associated with a specific predetermined chronic condition.

26. The predictive health management system according to claim 22, wherein the population information stored on the one or more non-transitory computer-readable storage media comprises age associated with each member of the population, and wherein the population information includes predetermined chronic conditions associated with at least some members of the population who are adults and includes predetermined chronic conditions associated with at least some members of the population who are children.

27. The predictive health management system according to claim 26, wherein the at least one member within the selected group is associated with population information for a child.

28. The predictive health management system according to claim 26, wherein the at least one member within the selected group is associated with a selected predetermined chronic condition.

29. The predictive health management system according to claim 21, wherein the set of predefined healthcare resources includes at least one healthcare resource selected from the group consisting of a hospitalization, a repeated hospitalization, or a readmission.

30. The predictive health management system according to claim 21, wherein the set of predefined healthcare resources includes at least one healthcare resource selected from the group consisting of an outpatient service, a laboratory service, a pharmacology service, or an ambulatory service.

31. The predictive health management system according to claim 21, wherein the selected threshold number of predetermined chronic conditions comprises three chronic conditions.

32. The predictive health management system according to claim 21, wherein the selected threshold number of predetermined chronic conditions comprises four chronic conditions.

33. The predictive health management system according to claim 21, wherein the selected threshold number of predetermined chronic conditions comprises five chronic conditions.

34. The predictive health management system according to claim 21, wherein the selected threshold number of predetermined chronic conditions comprises six, seven, eight, nine or ten chronic conditions.

35. The predictive health management system according to claim 21, wherein the set of predetermined chronic conditions comprises a plurality of chronic conditions, at least one of which being selected from the group comprising: hypertension, blood thinner/anticoagulant usage, COPD/asthma, skin ulcers, cellulitis, dementia, transplant, inflammatory bowel disease, sickle cell disease, rheumatic disease, hemophilia, muscular dystrophy, developmental delay, mental retardation, Down's syndrome, cystic fibrosis, Tay Sachs, cerebral palsy, uncontrolled seizures, autism, inherited diseases, schizophrenia, bipolar disease, substance abuse, congestive heart failure, diabetes, myocardial infarction, peripheral vascular disease, cerebrovascular disease, chronic pulmonary disease, connective tissue disease, ulcer disease, mild liver disease, hemiplegia, moderate or severe renal disease, diabetes with end organ damage, tumor, leukemia, lymphoma, moderate or severe liver disease, metastatic cancer, AIDs, depression, bipolar illness, or schizophrenia.

36. The predictive health management system according to claim 21, wherein the set of predetermined chronic conditions comprises mental illness or substance abuse, and wherein one or more processors configured, upon execution of the computer-readable executable instructions, to identify, via the one or more processors, the at least one member within the selected group having population information associated with mental illness or substance abuse as one of the sum of predetermined chronic conditions.

37. The predictive health management system according to claim 21, wherein the set of predetermined chronic conditions comprises skin ulcers or cellulitis, and wherein one or more processors configured, upon execution of the computer-readable executable instructions, to identify, via the one or more processors, the at least one member within the selected group having population information associated with skin ulcers or cellulitis as one of the sum of predetermined chronic conditions.

38. The predictive health management system according to claim 21, wherein the set of predetermined chronic conditions comprises the Charlson comorbidity index and wherein the predefined comorbidity value associated with each of the plurality of predetermined chronic conditions in the corresponds to those values in the Charlson comorbidity index.

39. The predictive health management system according to claim 21, wherein the representation of level of use of the at least one healthcare resource corresponding to healthcare services delivery for the at least one member within the selected group comprises a value corresponding to a risk of hospitalization, a repeated hospitalization or a readmission for the at least one member.

40. The predictive health management system according to claim 22, wherein the representation of level of use of the at least one healthcare resource corresponding to healthcare services delivery for the plurality of members within the selected group comprises a value corresponding to a risk of hospitalization, a repeated hospitalization or a readmission for the plurality of members.
